(19) United States
(12) Patent Application Publication
(10) Pub. No.: US 2025/0241845 A1
(43) Pub. Date: Jul. 31, 2025

(54) Facial Mask with Artemisia Argyi for Deeply Repairing Skin Problems and Using Method of the Facial Mask

(71) Applicants: Qinglin LAI, Fujian (CN); Xingwu LAN, Fujian (CN)

(72) Inventor: Qinglin LAI, Fujian (CN)

(73) Assignees: Qinglin LAI, Fujian (CN); Xingwu LAN, Fujian (CN)

(21) Appl. No.: 18/786,434

(22) Filed: Jul. 27, 2024

(30) Foreign Application Priority Data
Jan. 29, 2024 (CN) 202420204052.X

(51) Int. Cl. A45D 44/00; A61K 8/02; A61K 8/9789; A61Q 19/00

(52) U.S. Cl. CPC A61K 8/9789; A61K 8/0212; A61M 35/10; A61Q 19/00

(57) ABSTRACT

The present invention relates to a facial mask with Artemisia argyi for deeply repairing skin problems and a using method of the facial mask. The mask comprises a face towel cover body and a partition cloth, wherein the partition cloth is arranged in the middle of the face towel cover body to partition the face towel cover body into a first chamber and a second chamber; the first chamber is internally provided with a graphene heating layer; the second chamber is internally provided with a Chinese medicine layer of Artemisia argyi; the middle and lower parts of the face towel cover body are closed; and the upper part of the face towel cover body is open, and the face towel cover body is closed at the opening by a connecting element.

### CROSS-REFERENCE TO RELATED APPLICATIONS

This application claims the priority of China patent application No. 202420204052.X filed on Jan. 29, 2024, entitled “Facial mask with Artemisia argyi for Deeply Repairing Skin Problems”, the entire contents of which, including the amendments, are all incorporated herein by reference.

### TECHNICAL FIELD

The present invention relates to the technical field of beauty, in particular to a facial mask with Artemisia argyi for deeply repairing skin problems and a using method of the facial mask.

### BACKGROUND

Facial care has always been a topic of great concern. However, most products on the market solve superficial problems, and the deep-seated problems have not been solved, which leads to a rebound once the user stops using skin care products, and finally the user becomes dependent on skin care products.

In addition, the U.S. patent with a publication number of U.S. Pat. No. 20,210,106460A1 discloses a method for using beauty instrument with mask, which can be directly attached to a person's face through the facial mask without holding hands, thus freeing the hands of the user; through that control circuit of the controller, the skin of the face can be selectively stimulated without causing facial asymmetry; at least one heating layer is arranged on the flexible mask to heat the flexible mask. Meanwhile, U.S. Pat. No. 11,045,386 discloses a steam heating mask, by which the user can easily breathe, which actively generates steam heating in the mask, increases the absolute humidity in the mask, and improve the moist feeling of the throat and nasal mucosa by adopting the configuration of the steam generator in the area ratio of the main part of the mask and the adoption of a steam generator with a specific sheet configuration.

However, in the prior art and the patents disclosed above, the face is generally directly heated by a heating element, which only has the function of heating to promote the blood circulation of the face. In the face care, applying Artemisia argyi to the face is a common health care and medical method. The hot air of water can better open the pores of the skin, which is convenient for Artemisia argyi to better penetrate into the skin, and can warm the meridians, promote qi and blood circulation, dispel dampness and dispel cold, reduce swelling and dissipate stagnation, balance yin and yang, promote blood circulation and promote body metabolism, solve the problem at a deeper level; and at the same time, applying Artemisia argy to the face is also a method commonly used in health care and medical treatment to detoxify.

Based on the above problems, there is a need to put forward a facial mask of Artemisia argyi for deeply repairing skin problems. The facial mask of Artemisia argyicombins Artemisia argyi application with the facial mask by heating function, and the Artemisia argyi and the mask are heated by the heating layer, which can not only open pores to deeply clean and replenish moisture in the skin, but also allow the sexual tastes of Artemisia argyi and ginger to better enter the depths of the skin along with the hot air, so as to facilitate the better penetration of Artemisia argyi and ginger into the skin.

### SUMMARY

The present invention provides a facial mask with Artemisia argyi for deeply repairing skin problems, wherein the marks has a structure comprising a face towel cover body and a partition cloth, wherein the partition cloth is arranged in the middle of the face towel cover body to partition the face towel cover body into a first chamber and a second chamber; the first chamber is internally provided with a graphene heating layer; the second chamber is internally provided with a Chinese medicine layer of Artemisia argyi; middle and lower parts of the face towel cover body are closed, an upper part of the face towel cover body is open, and the face towel cover body is closed at an opening by a connecting element, so that the graphene heating layer and the Chinese medicine layer of Artemisia argyi are confined in an area covered by the first chamber and the second chamber.

The present invention further provides a facial mask with Artemisia argyi for deeply repairing skin problems, which includes a face towel cover body, wherein a cavity is arranged in the face towel cover body, an opening communicated with the cavity is arranged on an outer contour edge of the face towel cover body, a partition layer for partitioning the cavity into a first chamber and a second chamber is arranged in the face towel cover body, a heating layer is arranged in the first chamber, and a Chinese medicine layer with pharmacological effects is arranged in the second chamber; and
a connecting element for closing and opening the opening is arranged on the face towel cover body, so as to limit the heating layer and the Chinese medicine layer in an area covered by the first chamber and the second chamber.

The present invention further provides a use method of the facial mask, which includes the following steps:
providing a facial mask with Artemisia argyi for deeply repairing skin problems, wherein the facial mask with Artemisia argyi for deeply repairing skin problems includes a face towel cover body and a partition cloth, wherein the partition cloth is arranged in the middle of the face towel cover body to partition the face towel cover body into a first chamber and a second chamber; the first chamber is internally provided with a graphene heating layer; the second chamber is internally provided with a Chinese medicine layer of Artemisia argyi; middle and lower parts of the face towel cover body are closed, an upper part of the face towel cover body is open, and the face towel cover body is closed at an opening by a connecting element, so that the graphene heating layer and the Chinese medicine layer of Artemisia argyi are confined in an area covered by the first chamber and the second chamber;
the method includes the following steps:
adding or replacing the Chinese medicine layer of Artemisia argyi for the second chamber of the face towel cover body;
soaking the face towel cover body in hot water;
taking out the face towel cover body and squeezing out the moisture in the face towel cover body, so that the face towel cover body is kept in a wet and non-dripping state;
adding or replacing the graphene heating layer for a chamber of the face towel cover body;
using the graphene heating layer for heating, and when the Chinese medicine layer of Artemisia argyi is heated to a personal comfortable temperature, using a closer side of the face towel cover body for being applied to a face.

### BRIEF DESCRIPTION OF DRAWINGS

In order to explain the technical scheme of this application more clearly, the drawings needed in the implementation will be briefly introduced below. Obviously, the drawings described below are only some implementations of this application. For those skilled in the art, other drawings can be obtained according to these drawings without creative work.

FIG. 1 is a structural schematic diagram of a facial mask with Artemisia argyi for deeply repairing skin problems of the utility model;

FIG. 2 is an internal view of the face towel cover body of a facial mask with Artemisia argyi for deeply repairing skin problems of the utility model;

FIG. 3 is a schematic diagram of a graphene heating layer of a facial mask with Artemisia argyi for deeply repairing skin problems of the utility model.

### REFERENCE SIGNS

Face towel cover body (1); Partition cloth (2); First chamber (31); Second chamber (32); Connecting element (4); Graphene heating layer (5); Graphene heating sheet (51); Waterproof layer (52); Wire (53); Power connector (54); Control box (55); Nostril breathing channel (6); Strap (7); Chinese medicine layer of Artemisia argyi (8).

### DESCRIPTION OF EMBODIMENTS

In the following, the technical scheme in the embodiment of the application will be clearly and completely described with reference to the drawings in the embodiment of the application. Obviously, the described embodiment is only a part of the embodiment of the application, but not the whole embodiment. Based on the embodiments in this application, all other embodiments obtained by those skilled in the art without creative labor belong to the protection scope of this application.

Reference to “an example” or “an embodiment” herein means that a particular feature, structure or characteristic described in connection with an embodiment or an embodiment can be included in at least one embodiment of this application. The appearance of this phrase in various places in the specification does not necessarily refer to the same embodiment, nor is it an independent or alternative embodiment mutually exclusive with other embodiments. It is understood explicitly and implicitly by those skilled in the art that the embodiments described herein can be combined with other embodiments.

In this specification, for the sake of convenience, words and expressions indicating orientation or positional relationship such as “middle”, “upper”, “lower”, “front”, “rear”, “vertical”, “horizontal”, “top”, “inner” and “outer” are used to illustrate the positional relationship of constituent elements with reference to the attached drawings, only for the convenience of description. The positional relationship of the constituent elements is appropriately changed according to the direction of the described constituent elements. Therefore, it is not limited to the words and expressions described in the specification, and can be replaced appropriately according to the situation.

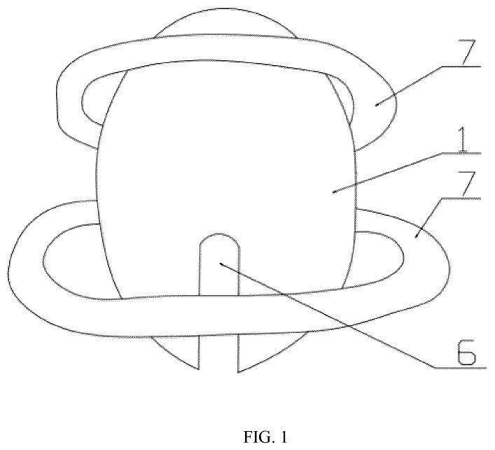

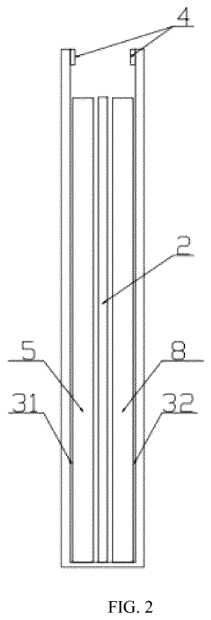

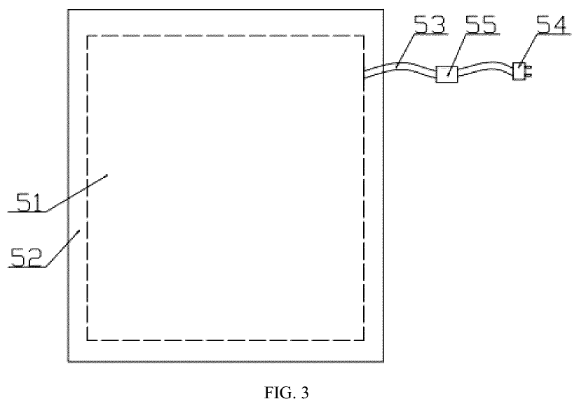

As shown in FIG. 1, FIG. 2 and FIG. 3, the present invention provides a facial mask with Artemisia argyi for deeply repairing skin problems, which structurally includes a face towel cover body 1 and a partition cloth 2, wherein the partition cloth 2 is placed in the middle of the face towel cover body 1 to partition the face towel cover body 1 into a first chamber 31 and a second chamber 32, wherein the face towel cover body 1 is adapted to the size of a human face and is fixed to the head by a strap 7; the first chamber 31 is internally provided with a graphene heating layer 5, and the second chamber 32 is internally provided with a Chinese medicine layer of Artemisia argyi 8; the middle and lower parts of the face towel cover body 1 are closed by sewing lines, and the upper part of the face towel cover body 1 is open, and the face towel cover body 1 is closed at the opening by a connecting element 4, so that the graphene heating layer 5 and the Chinese medicine layer of Artemisia argyi 8 are confined in the area covered by the first chamber 31 and the second chamber 32. With this structure, the graphene heating layer in the facial mask with Artemisia argyi for deeply repairing skin problems can heat the Chinese medicine layer of Artemisia argyi and the wet face towel cover body, thus opening the pores of the face to replenish the moisture of the skin; the sexual tastes of Artemisia argyi and ginger can better penetrate the skin along with the hot air to promote blood circulation and body metabolism; meanwhile, the flavor of Artemisia argyi can also enter the respiratory tract to warm the meridians of the heart, achieving two effects at the same time with the health care effect.

In some embodiments (not shown), the strap 7 can be designed as a velcro tape (hook and loop) fixed to the head or a lanyard fixed to the ear, and the strap can be an elastic belt or an elastic band. Among them, the velcro tape is a kind of closed device with stickiness and fluff, with one side being fine and soft fiber, and the other side being hard. When the two sides contact, they can stick together. The velcro tape is easy to open and close and easy to adjust. The use of elastic or elastic straps is not only comfortable to wear, but also suitable for occasions that need to be used close to the skin or head, and it is convenient to open and close without complicated operation.

As shown in FIG. 3, the graphene heating layer 5 includes a graphene heating sheet 51, and both sides of the graphene heating sheet 51 are covered with waterproof layers 52. The graphene heating sheet 51 is electrically connected with a wire 53, and the other end of the wire 53 is electrically connected with a power connector 54. The wire 53 is also electrically connected with a control box 55, and the control box 55 is provided with a power switch and a temperature regulating switch. With this structure, the power connector 54 and the wire 53 electrify and heat the graphene heating sheet 51, and then the heating temperature of the graphene heating sheet 51 is controlled by the control box 55; the control box 55 is provided with a power switch and a temperature regulating switch, which can control the on-off of the graphene heating sheet 51 and the power supply by the power switch, and adjust the heating temperature of the graphene heating sheet 51 by the temperature regulating switch; the waterproof layer 52 can prevent the graphene heating sheet 51 from being short-circuited after being immersed in water; after the graphene heating sheet 51 is heated, the far infrared rays generated by the graphene heating sheet 51 penetrate through the Chinese medicine layer of Artemisia argyi 8, so that the temperature of the Chinese medicine layer of Artemisia argyi 8 is increased by far infrared radiation, and Artemisia argyi and ginger can better enter the depths of the skin along with the hot air, which is convenient for the flavor of Artemisia argyi and ginger to better penetrate into the skin, and can promote the blood circulation of the human body to achieve the health care effect.

In some embodiments (not shown), the graphene heating layer can also be made of carbon fiber, metal fiber composite wire, copper foil or aluminum foil. Carbon fiber heating plate has high electrothermal conversion efficiency and good flexibility, which can meet the heating needs of various shapes and curved surfaces. Metal fiber composite wire heating sheet combines the high conductivity of metal and the flexibility of fiber, and has high bending resistance and stability. Copper foil and aluminum foil have good thermal conductivity.

As shown in FIG. 3, the power connector 54 is provided with a DC 5VType-C interface. With this structure, the power connector 54 supplies power to the typec power interface, and the graphene heating layer is electrified after supplying power to the power interface during use, so that the heating temperature of the graphene heating layer is automatically controlled between 41° C. and 45°° C., with a rated power of 4W and a peak power of 6W, which has the effects of infrared physiotherapy, health and soothing. In addition, the size of the Type-c interface is small, which can effectively prevent the invasion of water and dust, and improve the durability and stability of the equipment. The Type-c interface adopts standardized design and has good compatibility. This makes interoperability between different brands and devices possible, and users do not have to worry about interface incompatibility. At the same time, it also promotes the standardized production of various peripherals and accessories, and reduces the production cost and use cost.

In some embodiments (not shown), the power connector 54 is a DC (direct current) circular interface, a Mini USB interface and a Micro USB interface. The advantages of the DC interface are stability and reliability, which can provide stable current and voltage output and contribute to the normal work of equipment. Both Mini USB interface and Micro USB interface have the advantages of small interface, supporting hot plug and good compatibility.

The connecting element 4 is a mother-child patch arranged on the upper part of the face towel cover body 1. By adopting this structure, which is simple and convenient to use, the graphene heating layer 5 and the Chinese medicine layer of Artemisia argyi 8 are confined in the area covered by the first chamber 31 and the second chamber 32, which can prevent the graphene heating layer 5 and the Chinese medicine layer of Artemisia argyi 8 from slipping out of the face towel cover body 1. In addition, the use of the mother-child patch is very simple and quick, and it can be fixed with a single press, without complicated operation or tools, which greatly improves the use efficiency. Compared with the traditional adhesive or sewing method, the mother-child patch can be used repeatedly, which is not easy to be damaged, reduces the use cost and is more environmentally friendly. By adjusting the tightness of the mother-child patch, it can be ensured that the face towel cover body can provide the best comfort and fit in different use scenarios, and reduce the pressure on the face. The design of the mother-child patch makes the face towel cover body suitable for users with different facial shapes and sizes, which enhances the adaptability and versatility of the product. Because the mother-child patch is usually detachable, users can easily take it down for cleaning and replacement, and keep the face towel cover body clean and hygienic.

The connecting element 4 is a zipper arranged on the upper part of the face towel cover body 1. By adopting this structure, which is simple and convenient to use, the graphene heating layer 5 and the Chinese medicine layer of Artemisia argyi 8 are confined in the area covered by the first chamber 31 and the second chamber 32, which can prevent the graphene heating layer 5 and the Chinese medicine layer of Artemisia argyi 8 from slipping out of the face towel cover body 1. In addition, the zipper has smooth lines and simple and elegant appearance, which can improve the overall aesthetics of the face towel cover body. At the same time, the color and material of the zipper can also be coordinated with other parts of the mask to enhance the overall visual effect. There are many kinds of zippers, including core nylon zippers, double-bone zippers, invisible zippers, etc. Different zippers have different characteristics and applicable scenes. Therefore, the appropriate zipper type may be selected for the face towel cover body according to the needs to meet different use requirements.

In some embodiments (not shown), the connecting element 4 is a button and buttonhole arranged on the upper part of the face towel cover body, and a magnet bar arranged on the upper part of the face towel cover body. These connecting elements can realize quick connection and separation.

Because the mask shell and the heating layer in the prior art have an integrated structure, it is extremely inconvenient to clean after being used for a long time, and at the same time, it will damage the heating layer to a certain extent, affecting the use effect, and there will be certain safety hazards in the next use. By arranging the opening on the mask and opening and closing the opening through the connecting element, the heating layer and/or the Chinese medicine layer can be replaced, which is convenient for the next use.

The face towel cover body 1 is woven by micron slip yarns. By adopting this structure, the micro-spun yarn warps and micro-spun yarn wefts are interwoven, so that the face towel cover body 1 has good water absorption effect and excellent skin friendliness.

In some embodiments (not shown), the face towel cover body 1 can be woven from non-woven fabrics, cotton, polyester bamboo fibers and other materials besides micron slip yarns. These materials have the characteristics of softness, comfort, good moisture absorption and air permeability, and can be used for making face towel cover body.

As shown in FIG. 1, the face towel cover body 1, the graphene heating layer 5, the partition cloth 2 and the Chinese medicine layer of Artemisia argyi 8 are provided with nostril breathing channels 6; the width of nostril breathing channel 6 is 1-5 cm. By adopting this structure, a nostril breathing channel 6 of 1-5 cm is left on the face towel cover body 1 in the direction from the nostril to the jaw, so that the flavor of Artemisia argyi can enter the respiratory tract and lungs with hot air on the premise of ensuring breathing, and the meridians in the body can be better warmed.

In some embodiments (not shown), the nostril respiratory tract 6 can be designed as an adjustable nostril respiratory passage, covered with breathable material and covered with a mesh structure. Users can freely adjust the width of the channel according to their personal needs and comfort. This design can ensure that users can get the best breathing experience in different environments. Cover with highly breathable materials, such as microporous membrane or special breathable cloth, which can not only maintain the efficacy of Artemisia argyi, but also ensure smooth air circulation and reduce respiratory resistance. The design of the reticulate structure is adopted to form a plurality of tiny breathing holes. This design can not only keep the even distribution of Artemisia argyi, but also provide enough breathing space for air to pass smoothly.

The Chinese medicine layer of Artemisia argyi 8 includes a non-woven fabric patch, and two Chinese medicinal materials with pharmacological effects are filled in the non-woven fabric patch; wherein, the Chinese medicines are mixed Artemisia argyi and ginger powder. By adopting this structure, the non-woven fabric material can achieve the ventilation effect, so as to ensure that the flavor of Artemisia argyi and ginger in the Chinese medicine layer of Artemisia argyi can better enter the depths of the skin along the hot air under the action of hot compress, which is convenient for Artemisia argyi to better penetrate into the skin, can promote the blood circulation of the human body and achieve the health care effect. Wherein, ginger can help people delay aging and relieve deficiency and cold; improve the rough skin surface, help the body's stratum corneum metabolism, and soften the facial skin; accelerate the local blood circulation of the skin, dilute and prevent pigmentation; further, the rich vitamins contained in ginger have antioxidant effect and prevent pigment deposition in the body.

In some embodiments (not shown), the Chinese medicine layer 8 of Artemisia argyi includes a non-woven fabric patch, and beeswax, ginseng, angelica, angelica dahurica, poria cocos and the like are filled in the non-woven fabric patch. Beeswax, as a nutritious and astringent substance, is a high-grade natural product, which can improve the condition of subcutaneous tissue, activate its elastic fiber, prevent it from hardening, promote skin moistening and reduce wrinkles. In addition, beeswax also has the effect of removing the necrotic tissue and promoting granulation, which can increase skin elasticity and promote skin cell regeneration. Ginseng has strong antioxidant capacity, which can smooth wrinkles, promote blood circulation, accelerate metabolism, effectively regulate skin and restore its healthy and youthful state. In addition, ginseng can dilate skin capillaries, enhance cell vitality, improve the nutritional supply of hair follicles, and contribute to beauty and hair growth. Angelica sinensis has the effects of enriching blood, promoting blood circulation, regulating menstruation, relieving pain and moistening intestines. Its aqueous solution can inhibit tyrosinase activity, thus inhibiting the formation of melanin, and has a good effect on the treatment of pigmented skin diseases such as chloasma and freckles. In addition, angelica can make the face rosy and prevent the skin from being rough. Angelica dahurica is an important medicine for external use, which has the effects of expelling wind, relieving exterior syndrome, dispelling cold, relieving pain, removing dampness, inducing resuscitation, reducing swelling and expelling pus, and can improve human microcirculation, promote skin metabolism, eliminate excessive accumulation of pigments in tissues, and remove facial scars. Poria cocos has the effects of promoting diuresis and eliminating dampness, invigorating spleen and stomach, calming the heart and calming the nerves. “Compendium of Materia Medica” says that “Poria cocos can prolong life and resist aging, and make a user's face looks like a child's”, which shows that it has certain anti-aging effect.

The present invention further provides a facial mask with Artemisia argyi for deeply repairing skin problems, which includes a face towel cover body 1, wherein a cavity is arranged in the face towel cover body 1; an opening communicated with the cavity is arranged on the outer contour edge of the face towel cover body 1; a partition layer for partitioning the cavity into a first chamber 31 and a second chamber 32 is arranged in the face towel cover body 1; the first chamber 31 is internally provided with a heating layer; and the second chamber 32 is internally provided with a Chinese medicine layer with pharmacological effects; the partition layer is realized by using a partition cloth 2 arranged in the face towel cover body.

The face towel cover body 1 is provided with a connecting element 4 for closing and opening the opening, so as to limit the heating layer and the Chinese medicine layer in the area covered by the first chamber 31 and the second chamber 32.

The heating layer includes a graphene heating sheet 51, two sides of which are covered with waterproof layers 52. The graphene heating sheet 51 is electrically connected with a wire 53, and the other end of the wire 53 is electrically connected with a power connector 54. The wire 53 is also electrically connected with a control box 55, which is provided with a power switch and a temperature regulating switch. The Chinese medicine layer includes a non-woven fabric patch, and two Chinese medicines with pharmacological effects are arranged in the non-woven fabric patch. The connecting element 4 is a mother-child patch arranged on the face towel cover body 1 or a zipper arranged on the upper part of the face towel cover body. The face towel cover body 1 is provided with a nostril breathe channel 6 with a width of 1-5 cm. The face towel cover body 1 is woven by micron slip yarns. The face towel cover body 1 is provided with a strap 7 for being fixed to a head. The edge of the face towel cover body 1 is closed and connected by sewing lines.

The present invention further provides a use method of the facial mask, which includes the following steps:
providing a facial mask with Artemisia argyi for deeply repairing skin problems, which includes a face towel cover body 1 and a partition cloth 2, wherein the partition cloth 2 is arranged in the middle of the face towel cover body 1 to partition the face towel cover body 1 into a first chamber 31 and a second chamber 32, wherein the face towel cover body 1 has a size adapted to the size of a human face, and the face towel cover body 1 is fixed with a head through a strap 7; the first chamber 31 is internally provided with a graphene heating layer 5, and the second chamber 32 is internally provided with a Chinese medicine layer of Artemisia argyi 8; the middle and lower parts of the face towel cover body 1 are closed by sewing lines, and the upper part of the face towel cover body 1 is open, and the face towel cover body 1 is closed at the opening by a connecting element 4, so that the graphene heating layer 5 and the Chinese medicine layer of Artemisia argyi 8 are confined in the area covered by the first chamber 31 and the second chamber 32;
the method includes the following steps:
adding or replacing the Chinese medicine layer of Artemisia argyi 8 for the second chamber 32 of the face towel cover body 1;
soaking the face towel cover body 1 in hot water;
taking out that face towel cover body 1 and squeezing out the moisture in the face towel cover body 1, so that the face towel cover body 1 is kept in a wet and non-dripping state;
adding or replacing the graphene heating layer for the first chamber of the face towel cover body 1;
using the graphene heating layer for heating, and when the heated Chinese medicine layer of Artemisia argyi reaches a personal comfortable temperature, using a closer side of the face towel cover body to be applied to the face.

At the same time, there are two specific ways to use the facial mask with Artemisia argyi for deeply repairing skin problems in the above embodiments:

Usage 1: first, the Chinese medicine layer 8 of Artemisia argyi is put into the second chamber 32 near the face of the face towel cover body 1, then the face towel cover body 1 is put into a small amount of boiling water (the amount of boiling water shall submerge the face towel cover body 1) to soak for 3-5 minutes, then the face towel cover body 1 is taken out, without wringing out the water, so that some water is kept in the face towel cover body 1 (no dripping), and then the graphene heating layer 5 is placed in the first chamber 31 in the face towel cover body 1, and finally, the power switch is turned on, and the temperature is adjusted to a personal comfortable temperature through the temperature adjustment switch, so that the face application can be started.

Usage 2: first, the face towel cover body 1 is directly wetted, then the Chinese medicine layer of Artemisia argyi 8 and the graphene heating layer 5 are put into the first chamber 31 and the second chamber 32 of the face towel cover body 1, then the power switch is turned on, the temperature is adjusted to the highest through the temperature regulating switch to heat the Chinese medicine layer of Artemisia argyi 8 through the graphene heating layer 5, and when the smell of Artemisia argyi begins to float out, the heating temperature is adjusted to a personal comfortable temperature by adjusting the temperature regulating switch to start face application.

Some traditional moxibustion therapies burn Artemisia argyi sticks directly at human acupoints. However, due to the fragility of human face, it is difficult to directly moxibustion acupoints, and the doctors who need moxibustion have a high level. The demand for moxibustion in the field of family health care is even more difficult to meet. Another moxibustion therapy is to directly heat the dried Artemisia argyi with various heating technologies, and then moxibustion is carried out by thermal radiation. Although it is easier to operate than burning Artemisia argyi sticks, the air is too dry, which is easy to cause the problem of dry skin for people with dry face. Moreover, because the pores of the skin are not completely opened, Artemisia argyi is less likely to pass through the surface layer of the skin, therefore the effect is limited.

The facial mask with Artemisia argyi combines the Artemisia argyi face application with the facial mask through the heating function, and heats the Artemisia argyi and the mask through the heating layer, so that the pores can be opened for deep cleaning and the moisture of the skin can be replenished, and the flavor of Artemisia argyi and ginger can also better enter the depths of the skin along with the hot air, so that the properties of Artemisia argyi and ginger can better penetrate into the skin.

The technical means disclosed in the scheme of the present invention are not limited to the technical means disclosed in the above embodiments, but also include the technical scheme composed of any combination of the above technical features. It should be pointed out that for those skilled in the art, several improvements and embellishments can be made without departing from the principle of the present invention, and these improvements and embellishments are also regarded as the protection scope of the present invention.

What is claimed is:

1. A facial mask with Artemisia argyi for deeply repairing skin problems, wherein the marks has a structure comprising a face towel cover body and a partition cloth, wherein the partition cloth is arranged in the middle of the face towel cover body to partition the face towel cover body into a first chamber and a second chamber; the first chamber is internally provided with a graphene heating layer; the second chamber is internally provided with a Chinese medicine layer of Artemisia argyi; middle and lower parts of the face towel cover body are closed, an upper part of the face towel cover body is open, and the face towel cover body is closed at an opening by a connecting element, so that the graphene heating layer and the Chinese medicine layer of Artemisia argyi are confined in an area covered by the first chamber and the second chamber.

2. The facial mask with Artemisia argyi for deeply repairing skin problems according to claim 1, wherein the graphene heating layer comprises a graphene heating sheet, two sides of the graphene heating sheet are covered with waterproof layers; the graphene heating sheet is electrically connected with a wire, the other end of the wire is electrically connected with a power connector; and the wire is also electrically connected with a control box, and the control box is provided with a power switch and a temperature regulating switch.

3. The facial mask with Artemisia argyi for deeply repairing skin problems according to claim 2, wherein the power connector is provided with a DC 5VType-C interface.

4. The facial mask with Artemisia argyi for deeply repairing skin problems according to claim 1, wherein the connecting element is a mother-child patch arranged on the upper part of the face towel cover body.

5. A facial mask with Artemisia argyi for deeply repairing skin problems according to claim 1, wherein the connecting element is a zipper arranged on the upper part of the face towel cover body.

6. The facial mask with Artemisia argyi for deeply repairing skin problems according to claim 1, wherein the face towel cover body is woven by micron slip yarns.

7. The facial mask with Artemisia argyi for deeply repairing skin problems according to claim 1, wherein nostril breathing channels with a width of 1-5 cm are formed on the face towel cover body, the graphene heating layer, the partition cloth and the Chinese medicine layer of Artemisia argyi.

8. The facial mask for deeply repairing skin problems according to claim 1, wherein the Chinese medicine layer of Artemisia argyi comprises a non-woven fabric patch, and two Chinese medicines with pharmacological effects are filled in the non-woven fabric patch.

9. The facial mask with Artemisia argyi for deeply repairing skin problems according to claim 1, wherein the middle and lower parts of the face towel cover body are closed and connected by sewing lines.

10. The facial mask with Artemisia argyi for deeply repairing skin problems according to claim 1, wherein the face towel cover body is also provided with a strap for being fixed to a head.

11. A facial mask with Artemisia argyi for deeply repairing skin problems, comprising a face towel cover body, wherein a cavity is arranged in the face towel cover body, an opening communicated with the cavity is arranged on an outer contour edge of the face towel cover body, a partition layer for partitioning the cavity into a first chamber and a second chamber is arranged in the face towel cover body, a heating layer is arranged in the first chamber, and a Chinese medicine layer with pharmacological effects is arranged in the second chamber; and
a connecting element for closing and opening the opening is arranged on the face towel cover body, so as to limit the heating layer and the Chinese medicine layer in an area covered by the first chamber and the second chamber.

12. The facial mask with Artemisia argyi for deeply repairing skin problems according to claim 11, wherein the heating layer comprises a graphene heating sheet, and both sides of the graphene heating sheet are covered with waterproof layers; the graphene heating sheet is electrically connected with a wire, the other end of the wire is electrically connected with a power connector; the wire is also electrically connected with a control box, and the control box is provided with a power switch and a temperature regulating switch.

13. The facial mask with Artemisia argyi for deeply repairing skin problems according to claim 11, wherein the Chinese medicine layer comprises a non-woven fabric patch, and two Chinese medicines with pharmacological effects are filled in the non-woven fabric patch.

14. The facial mask with Artemisia argyi for deeply repairing skin problems according to claim 11, wherein the connecting element comprises a mother-child patch arranged on the face towel cover body.

15. The facial mask with Artemisia argyi for deeply repairing skin problems according to claim 11, wherein the connecting element comprises a zipper arranged on the upper part of the face towel cover body.

16. The facial mask with Artemisia argyi for deeply repairing skin problems according to claim 11, wherein a nostril breathing channel with a width of 1-5 cm is provided on the face towel cover body.

17. The facial mask with Artemisia argyi for deeply repairing skin problems according to claim 11, wherein the face towel cover body is woven by micron slip yarns.

18. The facial mask with Artemisia argyi for deeply repairing skin problems according to claim 11, wherein the face towel cover body is provided with a strap for being fixed to a head.

19. The facial mask with Artemisia argyi for deeply repairing skin problems according to claim 18, wherein the edges of the face towel cover body are closed and connected by sewing lines.

20. A method for using a facial mask, comprising:
providing a facial mask with Artemisia argyi for deeply repairing skin problems, wherein the facial mask with Artemisia argyi for deeply repairing skin problems comprises a face towel cover body and a partition cloth, wherein the partition cloth is arranged in the middle of the face towel cover body to partition the face towel cover body into a first chamber and a second chamber; the first chamber is internally provided with a graphene heating layer; the second chamber is internally provided with a Chinese medicine layer of Artemisia argyi; middle and lower parts of the face towel cover body are closed, an upper part of the face towel cover body is open, and the face towel cover body is closed at an opening by a connecting element, so that the graphene heating layer and the Chinese medicine layer of Artemisia argyi are confined in an area covered by the first chamber and the second chamber;
the method comprises the following steps:
adding or replacing the Chinese medicine layer of Artemisia argyi for the second chamber of the face towel cover body;
soaking the face towel cover body in hot water;
taking out the face towel cover body and squeezing out the moisture in the face towel cover body, so that the face towel cover body is kept in a wet and non-dripping state;
adding or replacing the graphene heating layer for a chamber of the face towel cover body; and
using the graphene heating layer for heating, and when the Chinese medicine layer of Artemisia argyi is heated to a personal comfortable temperature, using a closer side of the face towel cover body for being applied to a face.
